(19) United States
(12) Patent Application Publication
(10) Pub. No.: US 2024/0269346 A1
(43) Pub. Date: Aug. 15, 2024

(54) Implantable Device Comprising External Mobilisation Means for the Formation of Articular Cartilage

(71) Applicant: PALINGEN, SAINT-GEORGES-D'ORQUES (FR)

(72) Inventors: Didier POURQUIER, SAINT GEORGES D'ORQUES (FR); Didier MOUKOKO, SAINT MARTIN-DU-FOUILLOUX (FR)

(21) Appl. No.: 18/569,412

(22) PCT Filed: Jun. 16, 2022
(86) PCT No.: PCT/FR2022/051169

§ 371 Date: Dec. 12, 2023

(30) Foreign Application Priority Data
Jun. 17, 2021 (FR) FR2106453

(51) Int. Cl. A61L 27/36; A61L 27/38; C12N 5/077

(52) U.S. Cl. CPC A61L 27/3654; A61L 27/3612; A61L 27/3695; A61L 27/3817; A61L 27/3852; C12N 5/0655; A61L 2430/06

(57) ABSTRACT

The present invention relates to a removable implantable device intended for the production of articular cartilage, comprising: •a first support portion (2) made of biocompatible material, •a second support portion (3) made of biocompatible material and movably mounted on the first support portion (2), the first support portion (2) and the second support portion (3) defining therebetween a cavity that forms a cell-growth space provided for receiving osteochondrogenic cells which multiply in the cell-growth space, and operable external mobilisation means (5A, 5B) which can be configured to move the second support portion (3) relative to the first support portion (2) so as to generate shear inside the cell-growth space.

### FIELD OF THE INVENTION

The present invention relates to an implantable device intended to induce the formation of an articular cartilage, preferably suitable for the morphology of a deteriorated articular cartilage of an individual, in order to replace said section of deteriorated cartilage.

The invention relates, in particular, to an implantable device suitable for promoting the formation of specific cellular layers of the articular cartilage. The implantable device is combined with a cellular repair matrix. The repair matrix preferably comprises a periosteal graft.

### PRIOR ART

Articular cartilage damage (or “chondropathy”) is manifested by disintegration of one or more cartilaginous surfaces, which lose their mechanical properties for protection of bone surfaces and damping of mechanical stresses of a joint. Untreated chondropathy in humans can degenerate into advanced osteoarthritis, causing significant chronic pains and can be very disabling, in particular for elderly patients.

In the example of the knee, during natural walking or crouching movements, the pressure exerted on the knee joint between the femur and the tibia is very large. If the mobility and damping function of the articular cartilage is not maintained, these natural movements become painful.

Such lesions of the articular cartilage may be located on only one of the two articular surfaces which face one another, but both articular cartilaginous surfaces facing each other can also be affected. The source of articular lesions lies either wear of the cartilage linked to age (osteoarthritis), or in articular traumas linked to impacts or accidents, in particular in young subjects. In current medical practice, the degree of severity of chondropathy is evaluated clinically by the ICRS functional score, and radiologically (MRI) by the ICRS grade or by the Outerbridge classification.

The known prior art comprises numerous therapeutic approaches for patients suffering from chondropathy. In early forms of osteoarthritis, treatment is proposed based on anti-inflammatories by general administration or local injection. Intra-articular injections are also known, for example injections of hyaluronic acid (referred to as “viscosupplementation”) or plasma enriched with platelets. Relatively non-invasive therapeutic treatments under arthroscopy can also be proposed, such as articular washing with debridement, the Pridie microdrilling technique, the microfracture technique, or the use of three-dimensional matrices composed of collagen and hydroxyapatite.

However, the above-mentioned approaches provide results that are often temporary at the clinical level, and inconclusive for the formation of new cartilage. Histologically, the cartilage formed in this way is of poor quality.

Another known approach consists in directly implanting one or more healthy osteocartilaginous sections, from a joint of an individual to be treated, in the section of joint to be treated in the same individual (by “osteochondral autografting”). In this case, osteocartilaginous cores are collected, for example, from non-load bearing regions of the joint to be treated and are transferred to the section requiring a repair.

FIG. 1a shows an osteochondral autografting at a femoral condyle, for treating the knee joint of a human individual (Reprise du sport aprés autogreffe ostéochondrale en mosaïque des condyles fémoraux: 25 cas á 9 ans de recul moyen, Cognault, Seurat, Chaussard, Ionescu, Saragaglia, Revue de chirurgie orthopédique et traumatologique 101 (2015) pages 215-220).

FIG. 1a illustrates a “mosaicplasty” technique. A plurality of cores 13 (in this case four cores), taken beforehand from the same individual, are implanted, in a mosaic arrangement in the damaged joint surface. Each core 13 comprises a cartilaginous surface and an underlying thickness of subchondral bone. The cores 13 preferably have a diameter of approximately 7 or 8 millimetres and a length of approximately 15 millimetres.

However, the number of “donor” sites, in other words non-load bearing joint regions, suitable for collection of the cores, is limited. This technique of autografting by mosaicplasty therefore addresses the treatment of limited articular surface lesions (typically less than 3 square centimetres).

Another approach called “osteochondral allografting”, involves a healthy osteocartilaginous section from a different individual than the individual to be treated. This technique is suitable for treating larger surface articular lesions, using larger-volume osteocartilaginous grafts.

However, this technique is difficult to implement in current practice because the osteocartilaginous collection must be conducted on a deceased human body less than 12 hours after death.

FIG. 1b shows an example of an osteocartilaginous core 13bis intended, for example, for an articular osteochondral allografting. The core 13bis is shown isolated, after collection and before implantation in the patient. The length of the core 13bis is approximately 4 centimetres in this case.

Given the limits of the approaches presented above in relation to FIGS. 1a and 1b, it has been proposed to conduct an arthroplasty (implanting of an articular prosthesis) in the case of advanced osteoarthritis in humans. The joints frequently treated in this way include, in particular, the hip and knee joints. For advanced chondropathy of the knee, a final unicompartmental or total knee prosthesis is frequently surgically implanted, fixed permanently under anaesthesia.

The international patent application with publication number WO 2005/016175 A2 describes a final articular prosthesis designed in a rigid biocompatible material, implanted directly in the affected articular cavity, in order to restore the expected mechanical properties of the joint. However, in the case of such a permanent articular prosthesis, a long re-education of the joint must be implemented. Significant medical treatment in a specialised centre, comprising physiotherapy sessions and supportive care, is often necessary.

There is also a severe risk of loss of mechanical functionality of the articular prosthesis over time, through mechanical wear of the components of the prosthesis and/or through infection of the prosthetic material.

It results from the above that in the present state, current surgical practices and approaches do not enable an entirely satisfactory repair, of sufficient quality and durability, of the articular cartilage, and/or present risks of infection and/or mechanical wear of the implanted devices. In addition, some of these treatments from the known prior art require extensive medical and surgical treatment.

The known approaches do not therefore appropriately respond to public health challenges concerning the treatment of articular pain and articular mobility disorders linked to cartilage lesions.

### GENERAL DESCRIPTION OF THE INVENTION

There is therefore a need for an implantable device capable of promoting the generation of a new section of articular cartilage, in order to replace a region of deteriorated cartilage of a joint, in particular in the knee (femorotibial, or patellofemoral joint) or in the hip joint of human beings.

The desired device must enable the formation of a new osteocartilaginous tissue, advantageously comprising a section of articular cartilage and a layer of subchondral bone.

In order to obtain good quality osteocartilaginous tissue, the newly formed cartilage must include different layers of chondrocytes (superficial, transitional, radial and calcified cartilage layers) characteristic of the articular cartilage. These different functional layers of the articular cartilage are described, in terms of structure and cellular composition, in the following publication: Composition and Structure of Articular Cartilage: A Template for Tissue Repair, Poole, Kojima, Yasuda, Mwale, Kobayashi, Laverty, October 2001, Clinical Orthopaedics and Related Research 391(391):S26-S33.

The desired device must have excellent surgical manoeuvrability, and not require excessive medical intervention for its implantation. Preferably, the device must enable repair of larger surface articular regions in comparison with osteochondral autografting techniques, while being easy to implant.

Preferably, the desired device must induce the formation of a new section of cartilage, the geometry of which is well suited to the specific morphology of the joint to be treated. It is desired that the surface of the repaired cartilage has a geometry very close to the geometry of the native cartilage, which enables it to fulfil the geometrical criteria for optimum operation and to avoid new wear of the patient's cartilage after treatment.

A secondary objective is to provide a device enabling the production and installation of portions of the newly formed cartilage in two regions of damaged cartilage, on the two opposite faces of the same injured joint.

In order to respond to the above-mentioned needs, a first aspect of the invention relates to a removable implantable device intended for the production of articular cartilage, comprising:
a first support part made of biocompatible material,
a second support part made of biocompatible material, movably mounted on the first support part, the first support part and the second support part defining therebetween a cavity that forms a cell-development space,
the cell-development space being provided for receiving osteochondrogenic cells which multiply in the cell-development space,
and operable external mobilisation means configured to move the second support part relative to the first support part, so as to generate a shear inside the cell-development space.

The implantable device as defined above, is intended to enable the formation of an autologous osteocartilaginous tissue, shortly after having been implanted in an anatomical region that is preferably different from the joint to be treated.

This implantable device is removable and is implanted in a temporary manner in a tissular receiving space, preferably an intramuscular region or a subcutaneous region of the individual, which is therefore preferably separated from the joint to be treated.

One advantage of such a device is to enable, at the end of an external mobilisation phase of the device, the formation of an osteocartilaginous tissue with a geometry matching the surface of the cartilage needing to be repaired. The tissue formed can then be removed from the biocompatible supports which have served for its production and be implanted in the joint to be treated.

The osteocartilaginous tissue formed is, for example, implanted in the joint to be treated in a similar manner to the autografting or allografting techniques mentioned above.

Optionally and in a non-limiting manner, the implantable device according to this first aspect can have the following features, taken alone or in any technically feasible combination:
the device comprises a closed housing, formed by the first support part and the second support part, the cell-development space being defined inside the housing.
the first support part comprises an internal face facing the second support part, the second support part being configured to slide relative to the first support part along the internal face.
the second support part is movably mounted in translation relative to the first support part, in a direction parallel to an extension surface of the internal face.
the second support part is rotatably mounted relative to the first support part about an axis of rotation.
at least one from the first support part and/or the second support part comprises a cylindrical side face extending around the axis of rotation.
the support part further comprises a base substantially perpendicular to the side face, the external mobilisation means comprising a handle constrained to rotate with the base and rotatable around the axis of rotation.
the external mobilisation means comprise at least one mobilisation lace mounted on the second support part, configured to tow the second support part.
the mobilisation lace is mounted on a first external face of the second support part.
the device comprises an additional mobilisation link mounted on a second external face of the second support part, the second external face being opposite the first external face.
the mobilisation lace comprises a semi-rigid strand, preferably a strand made of polymer material.
the device comprises a slide configured to guide a movement of the second support part relative to the first support part.
at least one from the first support part and/or the second support part comprises a grid.
the grid defines a plurality of cell-growth cavities opening on an inner side of the device.
at least one from the first support part and/or the second support part comprises a network of rigidifying elements fixed to said support part.
a rigidity against shear at the rigidifying elements is strictly greater than a rigidity against shear in a central region of the cell-development space.

A second aspect of the invention relates to an implant for the formation of cartilage cells, the implant comprising:
an implantable device as defined above;
a cellular repair matrix, interposed between the first support part of the implantable device and the second support part of the implantable device, the cellular repair matrix comprising a volume of osteochondrogenic cells intended to multiply in the cell-development space of the implantable device.

Optionally and in a non-limiting manner, the implant according to this second aspect can have the following features, taken alone or in any technically feasible combination:
the cellular repair matrix comprises at least one periosteal graft.
the periosteal graft is vascularised.
the periosteal graft comes from a periosteum of the tibia.
the implant is configured to be removably implanted in an intramuscular region of an individual.
the implant is configured to be removably implanted in a subcutaneous region of an individual.

### GENERAL DESCRIPTION OF THE FIGURES

Other features, aims and advantages of the implant will emerge from the following description, which is given purely by way of illustration and not being limiting, and which should be read with reference to the attached drawings, in addition to FIG. 1a and FIG. 1b which have already been discussed:

FIG. 2 schematically illustrates three pathways for differentiating mesenchymal stem cells: to bone or fibrous or cartilage articular tissue, depending on the mechanical stresses exerted on the mesenchymal stem cells.

FIG. 3 schematically illustrates a plurality of successive implantation steps of an implantation according to an example of the invention, in an intramuscular region, then mobilisation by external links of said implant in order to promote the formation of new cartilage, then explanation of the device and transfer of the osteocartilaginous structure produced to the recipient joint.

FIG. 4a is a schematic perspective view from above of an implantable device according to a first exemplary embodiment, the device having the form of a parallelepiped housing, a first portion of the device and a second portion of the device being represented separately from one another. In this case, a periosteal pedicle graft is mounted on the first portion.

FIG. 4b illustrates the same implantable device viewed in the closed position, the first portion and the second portion being fitted together one on top of the other.

FIG. 5 is a schematic cross-sectional view (along the plane A-A shown in FIG. 4b) of the device of FIG. 4b, while a periosteal graft is disposed in the cell-development space.

FIG. 6 is a schematic longitudinal sectional view (in the plane B-B shown in FIG. 4a) of the device of FIG. 4b, illustrating a relative movement of the first support portion and the second support portion, as well as the development of osteochondrogenic cells.

FIG. 7 is a schematic cross-sectional view of the device of FIG. 4b after mechanical mobilisation of said implantable device, with production of two portions of osteocartilaginous tissue inside the device, one being convex and the other concave.

FIG. 8 schematically illustrates the separation of a first support part from a second support part and from two portions of newly formed osteocartilaginous tissue which are extracted from the two support parts, the implantable device conforming with the example of FIGS. 4a and 4b.

FIG. 9 is a schematic cross-sectional view of an implantable device according to a second exemplary embodiment, the device having the form of a parallelepiped housing with curves and being intended to produce a single portion of osteocartilaginous tissue with a concave surface at the upper element.

FIG. 10 is a schematic longitudinal sectional view of an implantable device according to a third exemplary embodiment, at the time of its implantation, the device having the general form of a cylindrical housing and being implanted in a muscle. Said device is intended to produce two portions of newly formed osteocartilaginous tissue having a flat shape, facing each other.

FIG. 11 is a schematic longitudinal sectional view of the device of FIG. 10 during its mechanical mobilisation, while the osteochondrogenic cells are growing.

FIG. 12 is a schematic longitudinal sectional view of an implantable device according to a fourth exemplary embodiment, the device having the general shape of a quasi-cylindrical housing, one base of which is convex and the other base concave. Said device is intended to produce a single portion of newly formed osteocartilaginous tissue with a convex surface at the lower element.

### DETAILED DESCRIPTION OF SEVERAL EMBODIMENTS OF THE INVENTION

The examples of implantable devices described below are preferably intended for the formation of a new cartilage which will be subsequently implanted in the knee (femorotibial, or patellofemoral joint), or in the hip joint, in particular in human beings.

The various examples of implantable devices described below are however adaptable, in order to promote the generation of a new section of cartilage for any articular surface of the human or animal body.

The examples below provide for the use of a periosteal graft as repair matrix suitable for generating osteochondrogenic cells, inside a space provided in the implantable device. As will be seen below, the periosteal graft used is preferably collected from the same individual and may or may not be vascularised.

However, it is noted that other types of cellular repair matrices that are known in tissue engineering can be used. The repair matrix may or may not be combined with substances or compositions assisting growth and directed differentiation to cartilage cells, such as proteins, growth factors, etc.

In all of the attached figures and throughout the description below, similar elements are given identical alphanumeric reference signs.

### General Principles Relating to the Formation of New Articular Cartilage

Articular cartilage is a flexible conjunctive tissue present in the joints, ensuring the mechanical functions of protecting the ends of bones, damping the pressure of loads exerted on the bone ends and harmonious sliding of the facing articular surfaces.

Articular cartilage mainly consists of cells called chondrocytes, and of an extracellular matrix (collagen, glycosaminoglycans). A function of the chondrocytes is synthesis and maintenance of the cartilaginous tissue. They are located in compartments which each contain one or more chondrocytes. These compartments are called chondroblasts. Sections of cartilage can be deteriorated by various pathologies affecting the cartilage. Natural repair of the articular cartilage is very inefficient. The development of cartilaginous lesions combines cartilage cracks, a reduction in its thickness, and lesions of the subchondral bone. This development can lead to a complete elimination of the cartilage with baring of the subchondral bone (eburnation).

Articular cartilage is present, in particular, at the epiphyseal surfaces of long bones. Chondrocytes come from the cellular differentiation of stem cells located in adults in the most superficial layer of the articular cartilage. Renewal of the chondrocytes is however very slow and this tissue has great difficulty repairing.

The long bones, with the exception of the cartilage regions, are covered by a membrane surface called the “periosteum”, ensuring bone repair in the event of fracture.

The periosteum is particularly rich in reparative stem cells for skeletal tissues (osteochondrogenic mesenchymal stem cells). For a detailed description of the composition and role of the periosteum, reference can be made to the following publication: Periosteum contains skeletal stem cells with high bone regenerative potential controlled by Periostin, Duchamp de Lageneste et at., Nature Communications, February 2018 22; 9(1):773.

Faced with the difficulty for the body of spontaneously repairing articular cartilage in damaged regions of the articular surface, the device proposed here aims to reconstitute, in the deteriorated area, an osteocartilaginous tissue that is qualitatively and geometrically equivalent to the native articular cartilage (as it was initially, before deterioration). The reconstitution of the osteocartilaginous tissue is realised by osteochondrogenic cells (typically from an autologous periosteal graft, as will be seen below).

The differentiation of mesenchymal stem cells to the different types of connective tissues depends, in particular, on the mechanical stresses in the environment of the mesenchymal stem cells. The study of these stresses falls within the more general context of “mechanobiology”, studying the influence of local mechanical factors on cellular differentiation pathways.

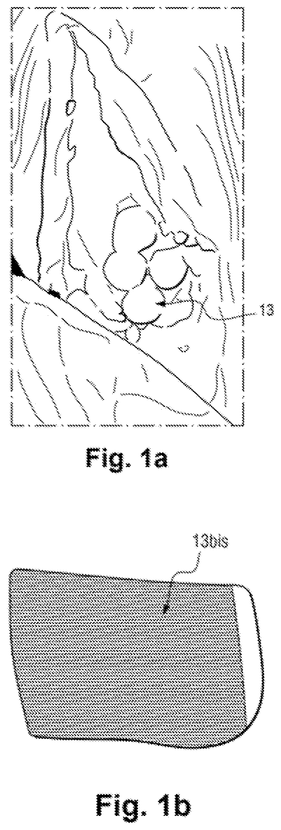

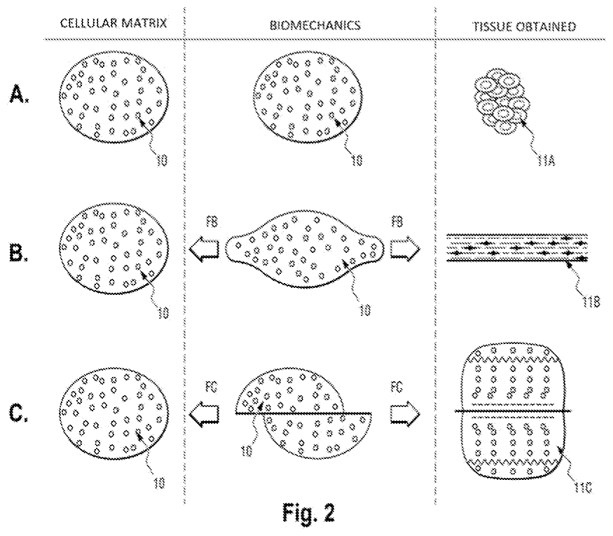

By way of illustration, attached FIG. 2 shows three pathways A, B and C for differentiation of mesenchymal stem cells.

Starting from a volume of still immature osteochondrogenic cells 10, it is possible to influence the differentiation pathway and therefore the phenotype and the future functionality of the newly formed tissue, through the mechanical stresses exerted in the cellular volume.

In “pathway A”, the cells are “free” and no mechanical stress (or minimal stress) is exerted. The osteochondrogenic cells then preferentially generate bone cells 11A (osteocytes). A clinical illustration of this pathway A is the future of the bone fracture site correctly stabilised by a cast or by an orthopaedic assembly. The fracture site (fracture callus), within which osteochondrogenic cells coming from the periosteum or from the bone marrow are present early, will differentiate into bone (in general after passing through the endochondral cartilage state).

On “pathway B”, stretching forces FB are exerted in a preferred direction. The formation of a fibrous tissue 11B (tissue constituting the ligaments) is then seen. A clinical illustration of this pathway B is, for example, the surgical reconstruction of the external lateral ligament of the ankle by a flap of periosteum taken from the fibula.

Finally, on “pathway C”, shear forces FC are exerted: the upper part of the volume of osteochondrogenic cells is moved to the left and the lower part of said volume is moved to the right. To the knowledge of the applicant, this differentiation pathway C promoting osteochondrogenesis has not been isolated to date, either naturally or in medical-surgical practice. The proposed device thus relies on shear movements applied to the cellular volume, promoting a directed differentiation of osteochondrogenic cells to the formation of chondrocytes, in order to ultimately obtain sections of articular cartilage.

After directed differentiation of the osteochondrogenic cells via pathway C, in this case new cartilaginous surfaces 11C are obtained, extending from one side to the other along the plane of cleavage.

The various examples of implantable device described below aim to push the directed differentiation of immature osteochondrogenic cells along a cellular differentiation pathway similar to the above-mentioned pathway C.

As will be seen below, the implantable device of the invention defines a cell-development space in which immature osteochondrogenic cells are induced to multiply. A repair matrix, typically comprising a (preferably autologous) periosteal graft is disposed in this space. Mechanical stresses are generated by external mobilisation, inside this space; generating a shear of the cellular repair matrix in a precise cleavage plane, identically reproducible with each movement.

In order to ensure the development of the osteochondrogenic cells and the directed differentiation of said cells to chondrocytes (pathway C), it is proposed here to install the implantable device, temporarily, directly in the body of the individual to be treated, in a tissue space favourable to this cell development.

The selected space (for example a muscular region or a subcutaneous region) nourishes the cellular repair matrix arranged inside the implantable device, in order to promote the development of osteochondrogenic cells. It is particularly relevant to implant the device in an environment capable of nourishing the cellular repair matrix, in the case where the latter comprises a non-vascularised periosteal graft.

Very preferably, an implantable device conforming to any one of the examples described below is not initially implanted in the joint to be treated or in the vicinity of the joint. It is proposed that said implantable device is installed, temporarily, in another region of the body of the individual.

It is very advantageous to removably install said implantable device in an intramuscular region of the individual, or in a subcutaneous region of the individual, during the growth and directed differentiation phase of the osteochondrogenic cells.

The intramuscular region has a biological environment that is particularly favourable to the growth of new osteochondrogenic cells. The following publication can be cited with regards to the interactions between the environment of the intramuscular region and the growth of osteochondrogenic cells: The potential role of muscle in bone repair, Liu, Schindeler, Little, J Musculoskelet Neuronal Interact 2010; 10(1) pp. 71-76, as well as the following publication: Role of muscle stem cells during skeletal regeneration, Rana Abou-Khalil, Frank Yang, Shirley Lieu, Anaïs Julien, Jaselle Perry, Catia Pereira, Frederic Relaix, Theodore Miclau, Ralph Marcucio, Céline Colnot, Stem Cells, May 2015, 33(5):1501-11. These two scientific publications show that the intramuscular region has not only high vascularisation which can supply an influx of blood nourishing the graft, but also contains progenitor cells contributing directly to bone repair.

It is thus advantageous to use a periosteal graft as repair matrix inside the implantable device, and to place the implantable device inside an intramuscular region. The combined action of the periosteum cells and the neighbouring myogenic cells forms a particularly suitable context for the multiplication and directed differentiation of osteochondrogenic cells, in order to form the osteocartilaginous tissue comprising the new section of cartilage and the subchondral bone.

Below, the main steps in the installing of the implantable device and obtaining of a new section of cartilage are described, according to an example.

### Installing an Implant and Mechanical Mobilisation in Order to Promote the Formation of Cartilage

The attached FIG. 3 shows the successive installation steps, in this case in an intramuscular region 80 of a muscle 8 of a human individual, for an implant comprising a removable implantable device 1 and comprising a cellular repair matrix 4, then the external mobilisation of said implant. Finally, newly formed cartilage 12 is extracted from the device 1 and the newly formed cartilage 12 is implanted in a section of articular surface (in this case, in the trimming cavity 92) of a section of damaged cartilage 90 of the same individual.

In this case, the implantable device 1 has the form of a parallelepiped housing. It conforms, for example, to any one of examples 1A and 1B described below. The same method steps could be implemented using another implantable device.

The articular surface to be treated is preferably separated from the muscle 8. In other words, there is not necessarily a link between the damaged articular surface to be treated and the muscle selected for installing the implantable device 1, except that the muscle very advantageously belongs to the same individual as the articular surface, in order to avoid potential rejection phenomena of the newly formed cartilage after implantation.

In the present example, the muscle 8 is a calf. The intramuscular region 80 corresponds, for example, to the central section of the calf.

In order to start the installation, the intramuscular region 80 is bared. An incision is, for example, made in the muscle 8. A space sufficient for inserting the device 1 is provided inside the muscle by the practitioner.

The device 1 is then inserted into the intramuscular region 80. The device 1 is combined with a repair matrix (not shown in FIG. 3) inserted in a cell-development space inside the device 1, forming an implant for the formation of cartilage. The device 1 is typically closed around the repair matrix.

One role of the repair matrix is to supply the osteochondrogenic cells intended, after multiplication and directed differentiation, to differentiate into chondrocytes and form a newly formed cartilage 12. Very preferably, the repair matrix comprises a periosteal graft.

The preferably autologous periosteal graft has, for example, been collected on a bone surface of the individual. In the present example, the periosteal graft is collected on the anterior face of the tibia. For example, the periosteal graft is a very thin rectangular flap, having a width for example between 1 centimetre and 5 centimetres and a length between 5 centimetres and 10 centimetres. Alternatively, the periosteal graft can come from any other bone of the individual. It may or may not be combined with a pedicle 44 (it is then referred to as a periosteum “flap”);

The device 1 is associated with external mobilisation means, enabling the movement of a first portion 2 of the device 1 to be forced relative to a second portion 3 of the device 1. A shear is thus generated between said two portions, in order to generate shear stresses within the cellular repair matrix.

In the present example, the external mobilisation means comprise mobilisation links 5. Here, a first pair of mobilisation links 5 is fixed to each side of the second portion 3, and a second pair of links is fixed to each side of the first portion 2.

This second pair of links makes it possible to stabilise the first portion 2 in place, relative to the intramuscular region, while the mobilisation links of the first pair of links are used to mobilise the second portion 3. In order to stabilise the links of the second pair relative to the intramuscular region (in particular during the mobilisation of the device 1), preferably, fasteners 53 fix said links against the skin. These fasteners 53 are preferably produced during closure of the incision of the muscle 8, for example by sutures.

It is possible to use more than two pairs of stabilisation or mobilisation links, if necessary.

Returning to FIG. 3, in order to enable subsequent mobilisation, after stitching of the intramuscular region 80, the mobilisation links 5 in this case have a subcutaneous internal part 52 and an external part 50 intended to remain outside the skin of the individual. Mobilisation links 5 of sufficient length are chosen in order to have external parts 50 of sufficient length to ensure easy manipulation.

Here, the mobilisation links 5 preferably comprise semi-rigid strands, preferably with plasticised threads and/or suture threads.

The incision of the muscle 8 is then closed so as to leave the external parts 50 outside. The external parts 50 pass through the incision points 51 made in the skin, preferably above the muscle 8. Then the fasteners 53 are produced, as appropriate.

The device 1 is made of one or more biocompatible materials, and the repair matrix is preferably autologous. The risk of rejection of the device 1 by the body is therefore limited.

At this stage, the implantable device 1 is preferably left immobile, remaining in the intramuscular region 80, in a context of muscle immobility. An advantage of this is to give the osteochondrogenic cells time to multiply within the cell-development space, inside the device 1. The osteochondrogenic cells then multiply without any mechanical shear stress, until the cell-development space is “colonised”.

In the following step, the device 1 is (preferably mechanically) immobilised in order to set the second housing portion 3 in motion relative to the first housing portion 2. Before mobilisation, the osteochondrogenic cells are generally in a relatively immature state, enabling their directed differentiation.

In the example illustrated in FIG. 3, during the external mobilisation phase:
The first housing portion 2 remains fixed relative to the muscle 8. For example, the mobilisation links 5 attached to the first portion 2 are held, in order to force the first portion 2 to remain fixed relative to the muscle.
The second housing portion 3 is moved relative to the muscle 8 and relative to the portion 2, via the mobilisation links 5 attached to the second portion 3. For example, a mobilisation link 5 located on one side of the second portion 3, is pulled for example in mobilisation direction D illustrated in FIG. 3, then alternately a mobilisation link 5 located on an opposite side of the second portion 3 is pulled.

It will be understood that the mechanical means for external mobilisation of the device 1 could be supplemented or replaced by alternative mobilisation means. For example, electromechanical and/or electromagnetic mobilisation means can be used. The second housing portion 3 can, for example, be motorised and controllable remotely.

An advantage of the use of electromechanical and/or electromagnetic mobilisation means, combined for example with a control unit incorporated in the implantable device 1, is to enable control of the external mobilisation of the device 1 with the help of control signals, which removes the need to pull on the mobilisation links during the mobilisation phase.

The external mobilisation of the device 1 is preferably controlled in real time by the practitioner using medical imaging techniques, for example by echography, so as to monitor the reality of the movements. Optionally, the device 1 can be equipped with one or more sensors for detecting a movement and/or a speed and/or an acceleration of the second portion 3 relative to the first portion 2. The device 1 comprises, for example, one or more accelerometers.

Preferably, the periosteal graft remains vascularised by a pedicle 44 (if a vascularised graft is used), in particular in the case where, during the external mobilisation of the device 1, the lower face of the portion 2 is immobilised.

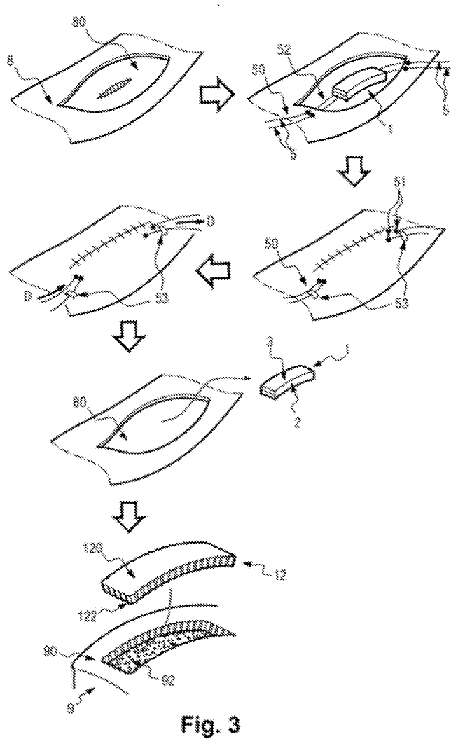

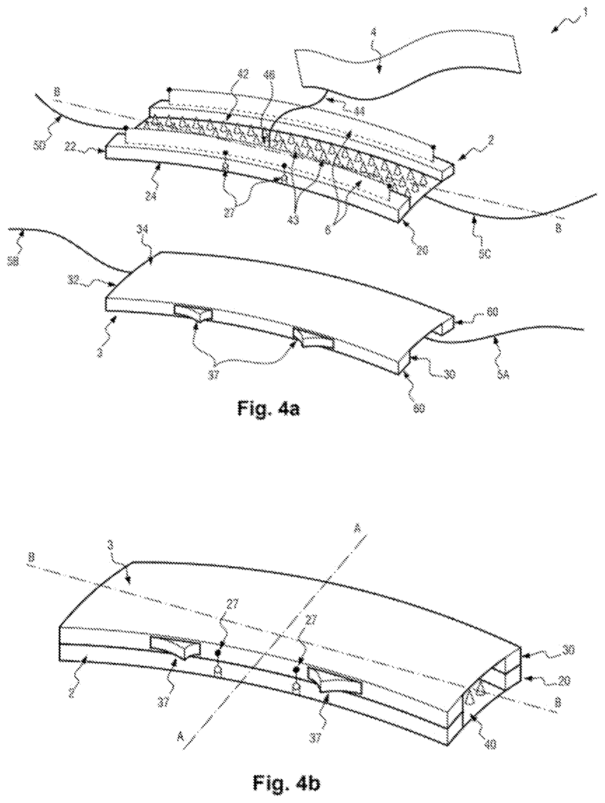

For this purpose, a through-hole 46 is preferably provided through the lower face of the first housing portion 2, so as to allow the vessels of the pedicle 44 of the periosteal graft to pass. Here, the through-hole 46 passes through the external wall 24 of the first portion 2, as shown in FIG. 4a. Here, the through-hole 46 is arranged in a central region of the grid 42 present on the first portion 2.

The through-hole 46 is preferably small. Indeed, the periosteal pedicle graft (which is preferably of low thickness and flexible) can be temporarily rolled up on itself, in order to slide through the small through-hole 46. The vascularised periosteum can then be unrolled and inserted in the cell-development space.

Alternatively, or in combination, a through-hole could be provided on the second portion 3 in order to allow the passage of a periosteal graft pedicle. However, it is advantageous that the vascular pedicle 44 passes through the first portion 2 which remains immobile relative to the muscular support, in order to avoid a clamping or a torsion of the vascular pedicle 44 due to movements of the second portion 3 during the mobilisation.

The external mobilisation phase of the implantable device 1 can be repeated as many times as necessary, during a period sufficient for generating shear stresses inside the device. For example, a mobilisation of several minutes can be carried out daily, during a period which is for example between one minute and ten minutes.

In a subsequent step, once the newly formed cartilage or cartilages 12 have been obtained in the cell-development space inside the device 1, the device 1 is removed from the body and disassembled, so as to recover the one or more newly formed cartilages 12.

The intramuscular region 80 is reopened in order to remove the device 1 from the muscle, then the first portion 2 is separated from the second portion 3 (an example of disassembly of a device 1 is described below in relation to attached FIG. 8).

As illustrated at the bottom of FIG. 3, the resulting newly formed cartilage 12 preferably comprises a cartilaginous surface 120 (in this case convex shaped) at the surface, and a region of newly formed subchondral bone 122 underneath the cartilaginous surface 120.

Also represented in FIG. 3 is an articular region 9 to be repaired, comprising the section of damaged cartilage 90.

The newly formed cartilage 12 is then implanted in place of the lesioned section, in the trimming cavity 92, after trimming of this cavity. In the present example, the articular region 9 is a femoral condyle. The joint treated is a femorotibial joint.

It is however understood that the newly formed cartilage could also be implanted in order to repair a damaged section of cartilage located in another joint. The installing of a newly formed cartilage is intended, in particular for the patellofemoral joint, the talocrural joint, the glenohumeral joint, the acromioclavicular joint and the coxofemoral joint.

The resulting newly formed cartilage 12 is, very advantageously, precisely matched to the geometry of the section of damaged cartilage 90 to be repaired. Thus, the newly formed cartilage 12 is precisely matched to the section of damaged cartilage 90.

Preferably, a trimming cavity 92 has been produced beforehand on the surface of the damaged section 90, the newly formed cartilage 12 then being implanted in the trimming cavity 92.

The term “trimming” shall mean a manoeuvre consisting of eroding the unhealthy cartilage and baring the subchondral bone. Trimming enables the damaged section of cartilage to be cleaned. Moreover, trimming can generate microbleeding, an influx of new blood vessels and local release of growth factors at the native subchondral bone.

The morphology of the newly formed cartilage 12 is controlled by the choice of the shape of the implantable device 1. Through the design of the device 1, and in particular as a function of the shape of the walls delimiting the cell-development space, it is possible to obtain a cartilage matched to a given specific morphology of the articular cavity. For this purpose, the first portion 2 and/or second portion 3 are preferably custom manufactured, as a function of the morphology of the damaged section 90 to be repaired.

Such custom manufacturing is, for example, performed by 3D printing. It is possible to use any 3D printing technique using biocompatible materials, for example one or more of the techniques described (in particular on page 22) in the publication Bioactive scaffolds for osteochondral regeneration, Deng, Chang, Wu, Journal of Orthopaedic Translation (2019) 17, pages 15-25, printing by extrusion, laser printing, stereolithography, fused deposition modelling (FDM), selective laser sintering (SLS), electrospinning, etc.

The 3D printing of the implantable device 1 can be based on a three-dimensional model taking account of the geometry of the section of damaged cartilage 90, obtained by medical imaging.

Examples of medical imaging techniques which can be used for this purpose are magnetic resonance imaging (MRI) (T2 relaxation time MRI or T1rho MRI) and/or delayed Gadolinium-Enhanced MRI of articular Cartilage (dGEMRIC) and/or standard radiography and/or an arthroscopy scanner.

After having recovered the one or more newly formed cartilages 12, said cartilage is implanted on the articular region 9. The installing of the newly formed cartilage 12 is optionally carried out by arthroscopy; such a procedure is minimally invasive.

A notable advantage of the approach proposed here is to allow the use of trimming of the section of damaged cartilage 90 (in this case from the surface of the femoral condyle) after obtaining the newly formed cartilage 12.

It is thus possible to control the quality and shape of the portion of newly formed cartilage 12, before trimming the lesioned region of the cartilage which must be repaired. It is also possible to adapt the shape of the trimming cavity 92 according to the shape obtained for the newly formed cartilage 12. It is also possible to abandon trimming, to the extent that it involves destroying the native cartilage still in place (although damaged), and the implantation procedure, in the event of insufficient production of newly formed cartilage inside the device.

Examples of implantable devices, intended for promoting the formation of a new cartilage, are describe below with reference to attached FIGS. 4a to 12. Removable implantable devices according to any one of these examples can be used to implement the procedure described above with reference to FIG. 3.

### Example 1A—Mobilisation in Translation in Order to Form Two Portions of Cartilage

FIG. 4a schematically illustrates a removable implantable device 1 according to a first exemplary embodiment, shown in perspective from above. The device 1 is preferably intended to be removably implanted in an intramuscular region, and/or intended to be removably implanted in a subcutaneous region.

The device 1 has the general form of a closed housing. The device 1 comprises a first housing portion 2 made of biocompatible material and a second housing portion 3 made of biocompatible material.

The first portion 2 is shown in the top part of FIG. 4a, and the second portion 3 is shown in the bottom part of FIG. 4a. In this FIG. 4a, the two housing portions are therefore separated from one another.

The first portion 2 and the second portion 3 are intended to define therebetween a cavity, which forms a cell-development space 40.

Attached FIG. 4b shows the same device 1, the first housing portion 2 being fitted together with the second housing portion 3 (for example via a slide 6, which has not been shown in FIG. 4b for better legibility of the figure). The cell-development space 40 is visible on one side, to the right of FIG. 4b.

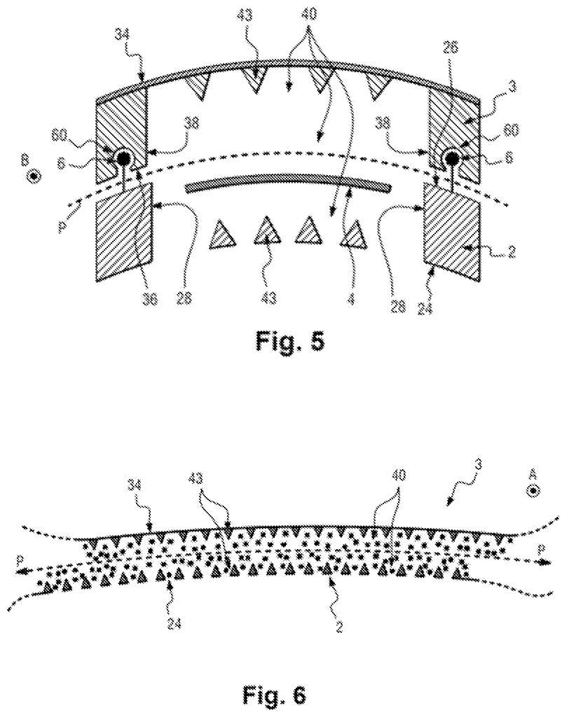

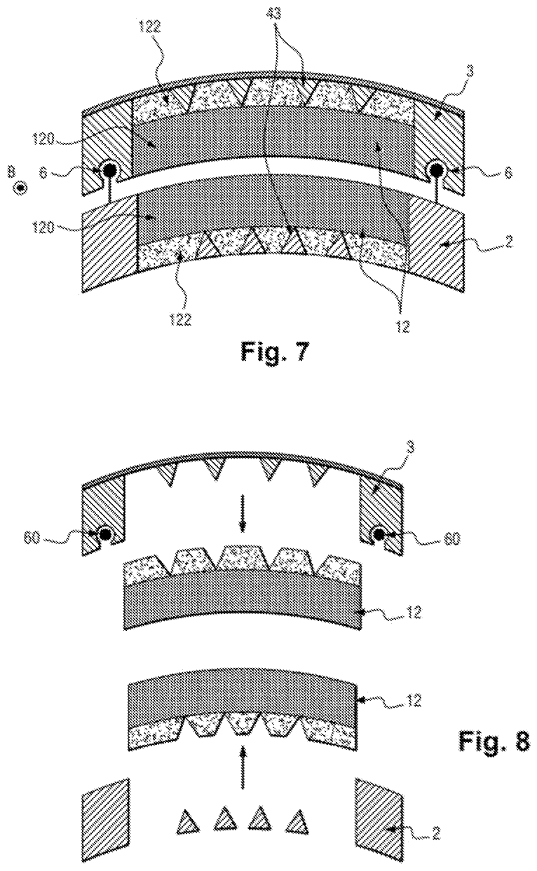

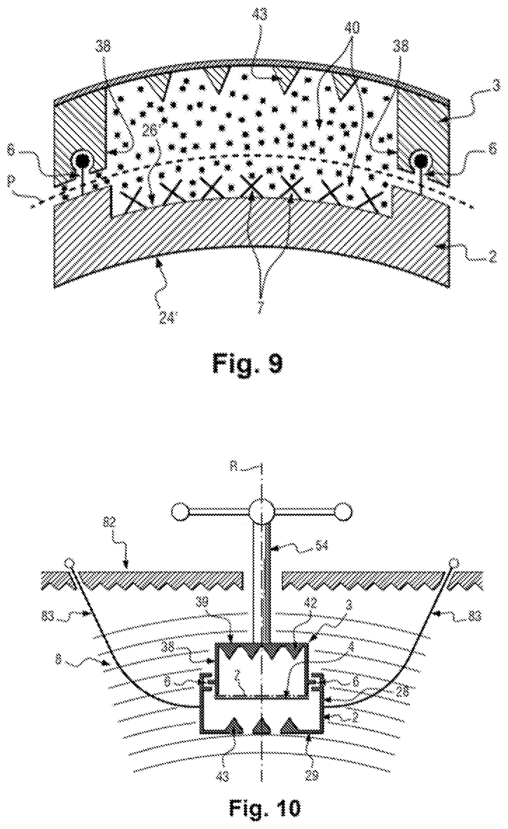

The inside of the device 1, intended to receive a cellular repair matrix 4, is also visible in cross-section in attached FIG. 5. In the cross-sectional view of this FIG. 5, and in FIGS. 7 and 9, the vertical gap between the internal face 26 of the first portion 2 and the opposite internal face 36 of the second portion 3 is not shown to scale, and has been enlarged for good legibility of these figures. Said vertical gap is very small (less than 1 millimetre, for example) compared with the dimensions of the first portion 2 and second portion 3.

The space 40 is intended to receive osteochondrogenic cells (in particular cells coming from the development of the cellular repair matrix 4) and to allow multiplication of these cells.

The second housing portion 3 is removably mounted on the first housing portion 2. The connection of the second portion 3 on the first portion 2 is sufficiently resistant not to give way during loading of the device 1 by the external mobilisation means (which will be described below) during the mobilisation phase.

The second portion 3 is movably mounted on the first portion 2; in the present example, the second portion 3 is movable in translation relative to the first portion 2 in a direction parallel to a longitudinal axis B of the housing.

The first portion 2 and/or the second portion 3 are preferably formed of a biocompatible material chosen from polymer materials (such as PTFE), extracellular matrices, bio ceramic materials, biological glasses or biocompatible metals, or any mixture of these materials.

The first housing portion 2 is at least partially laterally delimited by a first side face 20 and a second side face 22. The side faces 20 and 22 are substantially perpendicular to the longitudinal axis of the housing, and extend along two substantially parallel surfaces.

In the present example, the first side face 20 and the second side face 22 each have a central hollow. A hollow channel is thus formed along the longitudinal axis B of the first portion 2, connecting the side faces 20 and 22.

In addition, the first housing portion 2 is externally delimited by an external face 24, and internally delimited by an internal face 26. The external face 24 and the internal face 26 extend over two substantially parallel surfaces.

The internal face 26 is placed opposite the second housing portion 3. In the present example, the internal face 26 is hollowed out at its centre (at the hollow channel between the side faces 20 and 22) in order to allow the development of osteochondrogenic cells at the bottom of the first portion 2.

The first housing portion 2 further comprises two inner side walls 28 laterally delimiting the hollow channel. The inner side walls 28 extend towards the second portion 3, from a bottom wall located behind the external face 24.

The second housing portion 3 is laterally delimited by a first side face 30 and a second side face 32. The side faces 30 and 32 are substantially perpendicular to the longitudinal axis of the housing and extend over two substantially parallel surfaces. Here also, the first side face 30 and the second side face 32 each comprise a central hollow. An additional hollow channel is thus formed along the longitudinal axis B of the second portion 3, connecting the side faces 30 and 32.

The second housing portion 3 is externally delimited by an external face 34, and internally delimited by an internal face 36. The external face 34 and the internal face 36 extend over two substantially parallel surfaces. The internal face 36 is placed opposite the first housing portion 2.

The second housing portion 3 further comprises two inner side walls 38 laterally delimiting the hollow channel. The inner side walls 38 extend towards the second portion 2, from the bottom wall located behind the external face 34.

In the present example and as shown in FIG. 4b, when the housing constituting the device 1 is closed, the respective hollow channels of the first portion 2 and second portion 3 are placed facing one another.

Attached FIG. 6, which is a longitudinal sectional view of the device 1, corresponds to a sectional plane passing through said two hollow channels. FIG. 6 illustrates the first portion 2 and second portion 3 during the external mobilisation of the device 1. The cell-development space 40 extends inside the two hollow channels. Said space 40 is laterally delimited by the inner side walls 28 of the first portion 2, and by the inner side walls 38 of the second portion 3. The cell-development space 40 is also delimited from above (according to the orientation of FIG. 4a, by the inner side of the external face 34, and is delimited from below by the inner side of the external face 24.

Thus, when the second portion 3 is aligned with the first portion 2, the cell-development space 40 provided inside the device 1 has a generally parallelepiped form.

The function of the repair matrix 4 is to generate a multitude of osteochondrogenic cells. The repair matrix 4 advantageously comprises a periosteal graft, the biological function of which has been recalled above.

Very advantageously (but not compulsorily), the periosteal graft is a vascularised graft, for example a pedicled vascularised graft. FIG. 4a illustrates a periosteal graft attached to a pedicle 44, the pedicle 44 passing through the through-hole 46 provided in the body of the first housing portion 2. A non-vascularised graft is also possible, or optionally a combination of non-vascularised graft and vascularised graft in the case where at least two different grafts are used.

Advantageously, the periosteal graft results from autografting. In other words, the cells forming the periosteal graft are autologous cells, originating from the same individual in whom the articular implant is installed. The use of autologous cells promotes a much better tolerance of the osteochondrogenic cells, and eventually chondrocytes of the repaired cartilage, by the body of the individual. Thus, the regeneration of the cartilage is promoted more effectively and the risk of rejection is strongly limited.

In the present example, the periosteum graft comes from a periosteum of the tibia. An advantage is that the collection procedure on the bone of the tibia is easy and relatively non-invasive.

Preferably, before the action by the external mobilisation means, the repair matrix 4 takes the form of a cohesive solid element, not necessarily integral with the device 1. For example, the repair matrix 4 is inserted (and optionally sutured) in the cell-development space 40 just before insertion of the device 1 into the intramuscular or subcutaneous region.

An advantage of the through-hole 46 is to enable the vascularisation of the repair matrix 4 throughout the mobilisation phase by the external mobilisation means. The orifice enables passage of at least one blood vessel from the outside of the implant, in order that the repair matrix 4 is vascularised during the mobilisation. Such a vascularisation is very relevant in the case where the repair matrix 4 comprises a periosteal graft, in order to guarantee good cell development.

Thus, after installation of the implant (for example in the intramuscular region or the subcutaneous region), osteochondrogenic cells multiply in vivo from the repair matrix 4.

The view of FIG. 6 shows the osteochondrogenic cells filling the space 40 after their multiplication inside the implant. In the case where the first portion 2 and/or second portion 3 comprise conical tips 43, the osteochondrogenic cells preferentially fill the compartments defined by the tips 43.

In the present example, the second portion 3 is configured to slide relative to the first portion 2 along the internal face 26 during its movement in translation, by an external mobilisation of the device 1. The dashed line in FIG. 5 shows an extension surface P of the internal face 26.

The device 1 comprises external mobilisation means, enabling the movement of the second portion 3 relative to the first portion 2 to be forced. A shear is thus generated between the second portion 3 and the first portion 2, particularly in this case in the vicinity of the extension surface P of the internal sliding surface 26. Shear stresses are thus generated within the cell-development space 40.

Such shear stresses have the advantage of directing the differentiation of the cells present in the vicinity of the surface P in the cell-development space 40. More specifically, the cells closest to the surface P are the most exposed to the shear stresses. The cells will therefore preferentially differentiate into cartilage surface cells (differentiation pathway C illustrated in FIG. 2).

Thus, the external mobilisation of the device 1 creates a cleavage plane inside the cell-development space 40, in order to exert shear stresses.

The shear stresses thus created are similar to the stresses exerted on a mobility plane of a joint, in this case between a femoral condyle and a tibial plateau.

The device 1 is preferably mobilised after the osteochondrogenic cells have started to multiply (see the example illustrated in FIG. 6).

The external mobilisation means preferably comprise mechanical means. More preferably, said means comprise at least one mobilisation lace mounted on the second portion 3, configured in order to make it possible to tow the second portion 3 relative to the first portion 2.

The term “mobilisation lace” shall mean a fastener (thread, cable, belt, etc.) fixed on a part of the device 1, able to be pulled in order to force a relative movement of the second portion 3 relative to the first portion 2, in this case a movement in translation. Preferably, said mobilisation lace is then fixed on an outer face, typically on one of the side faces 30 and/or 32 of the second portion 3.

In the example of FIGS. 4a to 8, the external mobilisation means comprise a first mobilisation lace 5A fixed on the side face 30 and a second mobilisation lace 5B fixed on the opposite side face 32. Thus, the two mobilisation laces 5A and 5B are fixed on opposite outer faces of the second portion 3. These links have not been shown in FIG. 4b for better legibility.

As previously described in relation to FIG. 3, when the device 1 is implanted, the two mobilisation laces 5A and 5B preferably have a subcutaneous internal length of thread and an external length of thread intended to remain outside of the skin.

Using the mobilisation laces 5A and 5B, the second portion 3 can be towed from the left side and then from the right side (according to the orientation of FIGS. 4a and 6) relative to the first portion 2, so as to create a shear within the cell-development space.

The mobilisation laces 5A and 5B are preferably made with semi-rigid strands of thread. Said laces are preferably made of polymer material. It is possible, for example, to use any material usually used in suture threads.

Optionally and advantageously, the device 1 further comprises a slide 6 guiding the movement of the second portion 3 relative to the first portion 2. Thus, during loading of the device 1 by the external mobilisation means 5, the second portion 3 slides along the slide 6, so that the second portion 3 does not move in a transverse direction relative to the first portion 2.

Such a slide is visible in FIG. 4a and in FIG. 5. In the present example, the slide 6 has at least one monorail fixed in the first portion 2. The monorail is mechanically integral with the rest of the first portion 2. Said monorail in this case projects from the internal face 26 oriented facing the second portion 3. The second portion 3 then has, on its internal face 36, at least one groove 60 complementary to the monorail. For good legibility of the figures, the actual shape of the groove 60 is not illustrated in FIGS. 4a and 4b.

The slide 6 preferably has two identical longitudinal monorails on two sides, as shown in FIG. 4a.

Alternatively, a slide having a similar operation could be obtained by arranging one or two monorails on internal face 36 and one or two complementary grooves on internal face 26.

Such slides 6 are transposable to a variant of the device 1 where the external mobilisation means would be electrical and not mechanical.

Furthermore, the device 1 optionally comprises means for fixing the first portion 2 relative to the body, for example relative to the intramuscular or subcutaneous region in which the device 1 is present.

For this purpose, the device 1 preferably comprises at least one fixation link, still more preferably a pair of fixation links. In FIG. 4a, the device 1 comprises a first fixation link 5C on the side face 30, and further comprises a second fixation link 5D on the side face 32. These links have not been shown in FIG. 4b for better legibility.

The fixation link 5C and/or the fixation link 5D are for example attached to a muscle 8 in which the device 1 is implanted. The fixation links 5C and 5D are for example sutured to the muscle 8, so that the first portion 2 remains fixed while the second portion 3 is moved in translation during the external mobilisation phase. Alternatively, the fixation links 5C and 5D can have respective external lengths of thread intended to remain outside the skin, like the mobilisation laces 5A and 5B. The fixation links 5C and 5D can then be immobilised, for example manually, at these external lengths of thread.

The fixation links 5C and 5D are preferably made with semi-rigid strands of thread. Said links are preferably made of polymer material. It is possible, for example, to use any material usually used in suture threads.

The first portion 2 and/or second portion 3 preferably have a hollow structure, forming hollow recesses which extend the cell-development space 40.

In the example of FIGS. 4a to 8, the first portion 2 has a grid 42 forming such a hollow structure.

The grid 42 extends from the interior of the external face 24, in a direction of extension (width) perpendicular to the external face 24. For good legibility of the attached figures, the grid 42 has not been illustrated in FIGS. 4a and 4b.

The grid 42 defines, in the direction of the width (direction of extension perpendicular to the external face 24), a plurality of cell-growth through-cavities opening on an inner side of the device 1.

The cell-growth cavities are defined in this case between conical tips 43 formed in the grid 42. A base of the conical tips 43 is preferably oriented towards the outside and an apex of the conical tips 43 is oriented towards the inside.

Hollow through-compartments (for example parallelepiped and/or honeycomb compartments) are formed between the conical tips 43. The interior spaces of the compartments are located in the extension of the region where the repair matrix 4 is placed. Thus, the interior spaces of the compartments extend the cell-development space 40. The repair matrix 4 can then be installed and/or fixed above an upper surface of the grid 42 of the first portion 2, as illustrated in FIG. 5.

The growth cavities (in this case the compartments inside the grid 42) in this case pass through the two sides. On the outer side, the compartments pass through the external face 24 of the first portion 2, and on the inner side, the compartments join the cell-development space 40.

An advantage of such a grid 42 is that during the movement in translation of the second portion 3 relative to the first portion 2, the cells located in the vicinity of the external face 24 (which are the cells furthest from of the cleavage surface P) are mechanically more stable than the cells in the vicinity of the cleavage surface P.

During the external mobilisation of the implantable device, a gradient of rigidity against shear is thus created inside the cell-development space 40. The cells located in the vicinity of the external face 24 are induced to preferentially form bone cells after differentiation (pathway A of FIG. 2). It is thus possible to obtain layers of subchondral bone in the newly formed cartilage 12 obtained at the end of the mobilisation of the device 1.

Moreover, the grid 42 enables blood irrigation to reach the inside of the cell-development space 40, from outside the device 1.

In addition, an advantage of using conical tips 43 for forming the grid 42 is that the separating walls of the grid 42 then have a thickness at the base greater than their thickness at the surface, which accentuates the gradient of rigidity against shear.

In order to obtain the grid 42, one or more cavities can be natively provided in the design of the first portion 2. For example, a 3D model of the first portion 2 can incorporate the cavities of the grid 42 from its design. Alternatively, the cavities of the grid 42 can be obtained by perforation after production.

An additional advantage of providing a grid 42 at the first portion 2 is to enable intercommunication between the repair matrix 4 and the environment of the intramuscular region, and in particular a better vascularisation of the repair matrix 4.

In the present Example 1A, the second portion 3 does not comprise a grid. However, in this case, the second portion 3 comprises conical tips 43 forming growth cavities in the extension of the cell-development space 40. Thus, the conical tips of the second portion 3 are facing conical tips of the first portion 2. Unlike the first portion 2, the spaces between the conical tips 43 of the second portion 3 do not form through-perforations.

Alternatively, the second portion 3 could also be provided with through-perforations, for example similar to the grid 42. However, it is advantageous that the external face 34 is not perforated and that the cavities formed between the conical tips 43 of the second portion 3 do not pass through. Indeed, the second portion 3 is induced to move relative to the muscular region. The smooth external face 34 makes it possible to avoid friction or rubbing between the second portion 3 and the muscular environment during the movement of the second portion 3.

Optionally and advantageously, at their base, at least some of the conical tips 43 of the first portion 2 and/or of the second portion 3 can have recesses. The shape of the recesses of the conical tips 43 is preferably selected in order not to hinder the subsequent extraction of the newly formed osteocartilaginous tissue.

By way of example, such a recess can have a triangular shape at the base of the conical tip 43, and extend in a pyramid shape towards the apex of the cone (for example, in a three-sided pyramid shape). Thus, the triangular recess produced in the surface of the conical tip 43 preferably does not extend to the apex of said tip 43.

Such hollowed out conical tips 43 are advantageous, because they enlarge the total contact surface between the osteochondrogenic cells formed and the biomaterial support inside the cell-development space. Moreover, the rigidity of the first support portion 2 and/or of the second support portion 3 is locally increased. In order to simplify the attached figures, the conical tips 43 have not been shown with such recesses in these figures.

Alternatively, the conical tips 43 of the device 1 do not have recesses.

Preferably, means for blocking translational movement are provided on the first portion 2 and/or the second portion 3. The translation blocking means make it possible to define the range of movement in translation of the second portion 3 relative to the first portion 2.

In the present example, such translation blocking means comprise at least one protuberance 27 configured to abut against at least one wedge 37.

Here, the first portion 2 comprises a pair of protuberances 27 projecting upwards, on one side of the first portion 2. The protuberances 27 are preferably placed axially close to one another along the longitudinal axis B, on one side of the longitudinal axis B. Each protuberance 27 comprises, for example, a base and a rod projecting from the base with a spherical termination.

Here, the second portion 3 comprises a pair of wedges 37. The wedges 37 are placed facing one another, on the same side of the longitudinal axis B. The distance between the internal faces of the wedges 37 along the longitudinal axis B is preferably strictly greater than the distance between the protuberances 27 along the longitudinal axis B.

Thus, when the first portion 2 and the second portion 3 have been fitted together as illustrated in FIG. 4b, the protuberances 27 are inserted into their bases on the side face of the element 2 inside the longitudinal space provided between the wedges 37. The range of movement in translation of the second portion 3 relative to the first portion 2 is thus limited by the abutment of the protuberances 27 on the internal faces of the wedges 37.

It will be understood that alternatively the wedges and protuberances can be disposed on the other side, or both sides, and/or one or more protuberances can be provided on the second portion 3, and/or one or more wedges can be provided on the first portion 2.

At the end of the mobilisation of the device 1 by the external mobilisation means, which can optionally be repeated regularly during a mobilisation phase, the osteochondrogenic cells of the space 40 differentiate into cartilage cells and subchondral bone. Thus, in Example 1A, two facing portions of newly formed cartilage 12 are obtained, facing one another, as illustrated in attached FIG. 7.

At the surface of the grid 42, the cells closest to the surface P preferentially differentiate into cartilage cells, in order to obtain cartilaginous surfaces 120.

At the base of the grid 42 (in the vicinity of the external faces 24 and 34 on both sides of the housing), the cells furthest from the surface P preferentially differentiate into subchondral bone cells, in order to obtain subchondral regions 122.

FIG. 8 shows a disassembling of the device 1 after extraction of said device relative to the subcutaneous or intramuscular region. In the present example, a mechanical force is exerted in order to separate the first portion 2 and second portion 3. The slide 6 is thus broken, preferably at the monorail. For example, a base of the monorail remains on the first portion 2 and a distal part of the monorail is taken away by the second portion 3.

Thus, two integrated cartilages are obtained, ready to be assimilated in the articular cavity in order to repair the damaged cartilage sections.

In the present example, the damaged cartilage sections are located in a femorotibial joint. For example, one of the newly formed cartilages 12 obtained is position at a femoral condyle, and the other of the newly formed cartilages 12 is positioned on a tibial plateau facing the femoral condyle. Each newly formed cartilage is preferably embedded directly in the corresponding damaged section, for example in a respective trimming cavity.

The resulting newly formed cartilages 12 preferably conform to the geometries of the damaged cartilage sections to be repaired, and where applicable, conform to the geometries of the trimming cavities. After installation of the newly formed cartilage 12, the surface of the cartilage preferably extends continuously with the surfaces neighbouring the damaged section of cartilage.

The steps of mobilisation and separation of the portions of implantable device as described above, for the example of attached FIGS. 4a to 8, are transposed in similar manner to the other examples of removable implantable devices described below in relation to attached FIGS. 9 to 12.

An advantage of an implantable device according to the first example described above is to allow the simultaneous formation of two sections of newly formed cartilage 12, suitable for implantation in two damaged cartilage sections preferably belonging to two facing surfaces of a same joint.

The average total thickness of the cartilages of a human adult femorotibial joint is approximately 5 millimetres. Thus, an average thickness of the newly formed cartilage 12 (perpendicular to an extension surface of the outer face of the cartilage) is, for example, between 1 millimetre and 10 millimetres.

### Example 1B—Mobilisation in Translation with Single Cartilage Formation Compartment

FIG. 9 schematically illustrates an implantable device according to a second exemplary embodiment. The implantable device is viewed in cross-section, perpendicular to a longitudinal axis of extension.

The implantable device according to this second example has structural and functional features very close to the device 1 according to the first example described above, with the exception of the structure of the first housing portion 2. The second housing portion 3 is movable in translation relative to the first housing portion 2.

Unlike preceding Example 1A, the first portion 2 (which is preferably held fixed relative to the intramuscular region) is not perforated and does not comprise a grid 42. In this case, the first portion 2 comprises a solid internal face 26′, facing the second portion 3. In the case where a slide 6 is included, the internal face 26′ can extend between two longitudinal monorails of the slide 6.

Optionally and advantageously, a network of rigidifying elements 7 is disposed inside the first portion 2. The rigidifying elements 7 are placed on the inner side of the first portion 2, against the internal face 26′. The network of rigidifying elements 7 preferably covers a major part of the extent of the internal face 26′.

The rigidifying elements 7 have, for example, a cross shape, and are preferably fixed on the internal face 26′. A cross shape is obtained, for example, by 3D printing of biomaterial. Such rigidifying elements 7 are shown in FIG. 9.

An advantage of the rigidifying elements 7 is to increase the rigidity against shear at the bottom of the cell-development space 40, on the side of the first portion 2. Thus, the rigidity gradient is increased between the first portion 2 and the second portion 3.

In this second example, the periosteal graft preferably remains vascularised by a pedicle. For this purpose, a through-hole is preferably provided through the lower face of the first portion 2, in order to allow the vessels of the pedicle to pass.

The second portion 3 is movable in translation relative to the first portion 2. For example, a slide 6 is integrally fixed inside the first portion 2, in order to guide the movement along the surface P comprising the internal face 26′. During the mobilisation phase of the implantable device, the cells closest to the surface P tend to undergo a shear, while the cells at the bottom of the conical tips 43 tend to move with the second portion 3. The cells furthest from the surface P preferentially differentiate into subchondral bone.

After mobilisation and cellular development, a complete newly formed osteocartilaginous tissue, comprising a cartilage surface and a region of subchondral bone, is typically obtained inside the first portion 2 and enables the damaged articular section to be treated. After extraction of the implantable device according to this Example 1B from the body, the first portion 2 and the second portion 3 are separated from the osteocartilaginous tissue. The conical tips 43 are not incorporated in the newly formed osteocartilaginous tissue.

The cartilage potentially generated on the side of the second portion 3, in which the rigidifying elements 7 are embedded, is not re-used in the patient.

In a possible alternative, rigidifying elements 7 could be positioned on the side of the mobile second portion 3 (against the face 34), by replacing or in combination with the conical tips 43. The rigidifying elements 7 are then optionally separated from the second portion 3 at the time of the extraction of the newly formed osteocartilaginous tissue, and remain incorporated with this newly formed tissue. Incorporation of rigidifying elements 7 in the cartilage to be implanted is possible, in particular, if the rigidifying elements 7 are formed of a bioresorbable biomaterial, or a biomaterial that is very well tolerated over the long term inside the treated joint.

### Example 2A—Mobilisation in Rotation in Order to Form Two Portions of Cartilage

FIG. 10 schematically illustrates an implantable device according to a third exemplary embodiment. Said device is preferably intended to be removably implanted in an intramuscular region of an individual, and/or intended to be implanted in a subcutaneous region of an individual. FIG. 10 shows the device implanted inside a muscle 8, under the epidermis 82, viewed in cross-section.

This third example differs from the examples described above mainly through the rotary movement of the second housing portion 3 relative to the first housing portion 2, and through the structure of the first portion 2 and the second portion 3.

In this case, the first portion 2 is generally cylindrical around the axis R, open at the bottom (according to the orientation of FIG. 10). The first portion 2 comprises a side wall 28 and a bottom wall 29. The bottom wall 29 is preferably disc-shaped. The side wall 28 extends downwards from an annular edge of the bottom wall 29.

The second portion 3 also has a generally cylindrical shape around the axis R, open at the top (according to the orientation of FIG. 10). The second portion 3 comprises a side wall 38 and a bottom wall 39. The bottom wall 39 is disc-shaped. The side wall 38 extends upwards from an annular edge of the bottom wall 39.

The second portion 3 is removably mounted on the first portion 2.

The first portion 2 and/or the second portion 3 preferably have a rotational symmetry around the axis R. The first portion 2 and the second portion 3, closed on one another, thus form a generally cylindrical closed housing.

The external diameter of the side wall 38 is advantageously less than the internal diameter of the side wall 28, so that the second portion 3 is rotatable around of the axis R inside the side wall 38.

In a similar manner to the two preceding examples, the second portion 3 is configured to be moved relative to the first portion 2, in particular under the effect of an external mobilisation.

The device preferably comprises a slide 6 intended to guide the rotation of the second portion 3 around the axis R relative to the first portion 2.

In the example of FIGS. 10 and 11, the slide 6 comprises an annular rib extending radially from the outer side (relative to the axis R) from the side wall 38 of the second portion 3, preferably in the vicinity of a free edge of the side wall 38. The first portion 2 then comprises an annular groove radially oriented towards the interior (relative to the axis R) from the side wall 28. It will be understood that, alternatively, a slide can be formed by an annular rib of the first portion 2 directed inwards, complementary to the groove of the second portion 3.

Optionally, the device comprises additional means for fixation and guidance in rotation of the second portion 3 relative to the first portion 2.

By way of example, the free edge of the side wall 28 (directed towards the second portion 3) can have external threads on the radially outer side. During the mounting of the second portion 3 on the first portion 2, in order to obtain the closed housing, an additional fixation ring of diameter greater than the outer diameter of the first portion 2 can be attached over the slide 6 and closed on the free edge of the side wall 28. The fixation ring then preferably has, on the radially inner side, internal threads complementary to the external threads of the side wall 28, making it possible to screw said ring against said wall. Such a fixation ring is not shown in FIGS. 10 to 12.

A cell-development space 40 is defined axially between the inner side of the bottom wall 29 and the inner side of the bottom wall 39. This cell-development space 40 can receive a repair matrix 4. This matrix has the features already described above. Said matrix preferably comprises, in particular, a periosteal graft. Preferably, again in this third example, the periosteal graft remains vascularised by a pedicle. For this purpose, the first portion 2 and/or the second portion 3 can comprise a through-hole for the pedicle (not shown in the attached figures).

As in the preceding examples, the function of the repair matrix 4 is to generate a multitude of osteochondrogenic cells in the cell-development space 40. Preferably, after a certain cell-development time, the osteochondrogenic cells fill the interior space of the first portion 2 and the interior space of the second portion 3, as illustrated in FIG. 11.

In a similar manner to the two preceding examples, external mobilisation means 5 are operable in order to move the second portion 3 relative to the first portion 2, so as to generate a shear within the cell-development space 40.

In the present example, the external mobilisation means 5 comprise a handle that can be rotated around the axis of rotation R. In this case the movable handle is fixed to the bottom wall 39. Here, the movable handle comprises a sleeve 54 extending from the bottom wall 39 along the axis R and a gripping part 56 (in this case in the form of a wheel) extending towards the outside from the sleeve 54.

The movable handle is integral with the second housing portion 3, in its rotation around the axis R. Thus, if a practitioner or if the patient takes hold of the gripping part 56 and pivots said part as illustrated in FIG. 11, the second portion 3 is rotated.

Preferably, during the mechanical action of the second portion 3, the first portion 2 remains fixed relative to the muscle 8. Stabilisation means of the first portion 2 are, for example, provided for this purpose, such as fasteners 83.

The osteochondrogenic cells present in the volume located radially inside the side wall 38 tend to be driven by the rotary movement of the second portion 3, while the osteochondrogenic cells present in the volume located radially inside the side wall 28 tend to remain fixed.

A shear surface Z is shown at the interface between the space inside the side wall 28 and the space inside the side wall 38. During the rotary movement of the second portion 3, shear stresses are generated, in particular at the cells located in the vicinity of the shear surface Z.

As described above, such shear stresses promote a directed differentiation of the osteochondrogenic cells into cartilage cells. The development of cartilaginous surfaces is promoted close to the shear surface Z.

Optionally and advantageously, in order to create a gradient of rigidity against shear inside the space 40 (high rigidity against shear in the vicinity of the bottom walls 29 and 39, and lower rigidity close to the shear surface Z), the first housing portion 2 and/or the second housing portion 3 comprise conical tips 43 extending from the respective bottom walls 29 and 39 of said two housing portions. The conical tips 43 form compartments therebetween, for example in the form of a parallelepiped or honeycomb-shaped. The cell-development space 40 extends to the bottom of the compartments.

In the present example, conical tips 43 are provided both in the first portion 2 and in the second portion 3. An advantage is to increase the gradient of rigidity against shear inside the cell-development space 40. Moreover, the conical tips 43 form cavities which extend the cell-development space 40 between the first portion 2 and the second portion 3. Thus, the formation of a subchondral region is promoted in the newly formed cartilage obtained.

On the first fixed portion 2, the cavities formed between the conical tips 43 are preferably through-cavities. Thus, a grid 42 is formed on the bottom wall 29, which is induced to remain fixed.

By contrast, the bottom wall 39 is preferably not perforated. The external surface of the bottom wall 39 is preferably smooth.

Optionally and advantageously, the implantable device further comprises stabilisation means of the first portion 2 relative to the region of the body in which the device is implanted, in this case the muscle 8. In this example, the device comprises fasteners 83. The fasteners 83 fixedly connect the first portion 2 (for example the side wall 28) to the epidermis 82. The fasteners 83 comprise, for example, two suture threads each fixed at one end on the side wall 28, and at the opposite end of the epidermis 82. Thus, during the mobilisation of the second portion 3, the first portion 2 remains fixed relative to the muscle 8.

An advantage of an implantable device according to this third example is that the first portion 2 and the second portion 3 are not moved axially relative to one another during the mobilisation. The two volumes of osteochondrogenic cells respectively comprised inside the first portion 2 and the second portion 3 remain in contact throughout the mobilisation. This makes it possible to avoid the shear stresses being insufficient at the edges.

The implantable device according to this third example enables simultaneous formation of two cartilages. After extraction of the newly formed osteocartilaginous tissues, the conical tips 43 (if present) are preferably not incorporated with the newly formed tissues.

In a possible alternative, the implantable device of this Example 2B can comprise one or more networks of rigidifying elements 7 inside the housing. The rigidifying elements 7 of similar structure to that described above are, for example, positioned against the bottom wall 29 and/or against the bottom wall 39, and preferably have a cross shape. The rigidifying elements 7 preferably replace the conical tips 43 and cover a major part of the extent of the corresponding bottom wall. Such rigidifying elements 7 can be incorporated in the newly formed osteocartilaginous tissues (in particular if these rigidifying elements are formed of bioresorbable material or of another material that is well-tolerated over the long term by the joint).

### Example 2B—Mobilisation in Rotation in Order to Form a Convex Cartilage

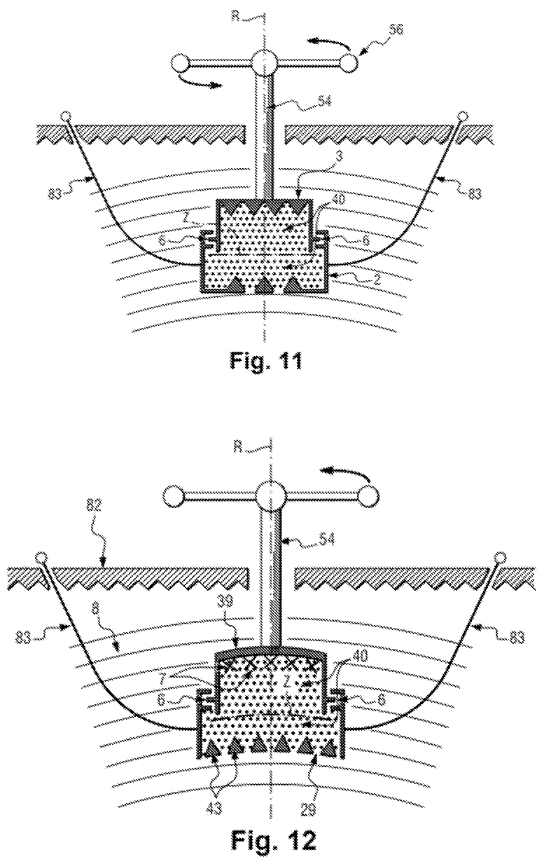

FIG. 12 shows an implantable device according to a fourth example, structurally and functionally similar to the third example.

In this fourth example, the second portion 3 is rotatable around the axis R relative to the first portion 2, via external mobilisation means 5. The device of the fourth example differs from the device of the third example through certain structural features of the first portion 2 and of the second portion 3. For the mounting of the second portion 3 on the first portion 2, as in the preceding example, an adjustable threaded fixation ring can be provided on the second portion 3.

In this fourth example, the bottom wall 39 of the second portion 3 is preferably smooth and not perforated. The bottom wall 29 of the first portion 2 is preferably perforated, so as to form a grid 42 promoting the vascularisation of the repair matrix 4.

In this fourth example, the periosteal graft preferably remains vascularised by a pedicle. For this purpose, a through-hole is preferably provided through the first portion 2, in order to allow the vessels of the pedicle to pass.

In this case, the bottom wall 29 of the first portion 2 and/or the bottom wall 39 of the second portion 3 do not have a flat geometry. Here, the two bottom walls 29 and 39 are curved. A convexity of the bottom walls 29 and 39 preferably corresponds to a convexity of the damaged section of cartilage to be repaired. The convexity necessary for the shape of the bottom walls 29 and 39 is determined, for example, from a 3D model of a section of cartilage to be treated. An advantage is to provide, from the design of the implantable device, an appropriate final shape for the newly formed cartilage obtained at the end of the mobilisation phase.

It will be understood that such an adaptation of the convexity of the mobile portions is likewise applicable for any of the other examples described above.

In addition, in this case the implantable device of this Example 2B comprises rigidifying elements 7 positioned against the bottom wall 39. The rigidifying elements 7 preferably replace the conical tips 43 of the second portion 3 of the preceding Example 2A. An implantable device according to the fourth example is suitable for enabling the formation of a single newly formed cartilage on the side of the first portion 2, in order to treat a single damaged section of cartilage having, for example, a non-flat shape (convex shaped in the present Example 2B).

1-18. (canceled)

19. A device comprising:
a first support made of biocompatible material;
a second support made of biocompatible material and movably mounted on the first support, the first support and the second support defining therebetween a cavity; mobilisation members configured to move the second support relative to the first support so as to generate shear inside the cavity.

20. The device of claim 19, wherein at least one of the first support and the second support comprises a bottom wall and two side walls extending from the bottom wall, wherein the two side walls delimit the cavity.

21. The device of claim 19, wherein the device comprises a closed housing formed by the first support and the second support, wherein the cavity is defined inside the housing.

22. The device of claim 19, wherein the first support comprises an internal face facing the second support, wherein the second support is configured to slide relative to the first support along the internal face.

23. The device of claim 22, wherein the second support is movably mounted in translation relative to the first support in a direction parallel to an extension surface of the internal face.

24. The device of claim 19, wherein the second support is rotatably mounted relative to the first support around an axis of rotation.

25. The device of claim 24, wherein at least one of the first support and the second support comprises a cylindrical side face extending around the axis of rotation.

26. The device of claim 25, wherein the at least one of the first support and the second support further comprises a base substantially perpendicular to the cylindrical side face, wherein the mobilisation members comprise a handle constrained to rotate with the base and rotatable around the axis of rotation.

27. The device of claim 19, wherein the mobilisation members comprise at least one mobilisation lace mounted on the second support and configured to tow the second support.

28. The device of claim 27, wherein the at least one mobilisation lace is mounted on a first external face of the second support, wherein the device further comprises an additional mobilisation link mounted on a second external face of the second support, the second external face being opposite the first external face.

29. The device of claim 27, wherein the at least one mobilisation lace comprises a semi-rigid strand, preferably a strand made of polymer material.

30. The device of claim 29, wherein the at least one mobilisation lace comprises a strand made of polymer material.

31. The device of claim 19, further comprising a slide configured to guide a movement of the second support relative to the first support.

32. The device of claim 19, wherein at least one of the first support and the second support comprises a grid, wherein the grid defines a plurality of cavities opening on an inner side of the device.

33. The device of claim 19, wherein at least one of the first support and the second support comprises a network of rigidifying elements fixed to the at least one of the first support and the second support, wherein a rigidity against shear at the rigidifying elements is strictly greater than a rigidity against shear in a central region of the cavity.

34. An implant comprising:
the device of claim 19; and
a matrix interposed between the first support and the second support, the matrix comprising a volume of osteochondrogenic cells.

35. The implant of claim 34, wherein the matrix comprises at least one periosteal graft, preferably a vascularised periosteal graft.

36. The implant of claim 34, wherein the matrix comprises a vascularised periosteal graft.

37. The implant of claim 36, wherein the vascularised periosteal graft comes from a periosteum of the tibia.

38. The implant of claim 34, further configured to be removably implanted in an intramuscular region of an individual and/or a subcutaneous region of an individual.
